(19) United States
(12) Patent Application Publication
(10) Pub. No.: US 2007/0249632 A1
(43) Pub. Date: Oct. 25, 2007

(54) PHARMACEUTICAL COMPOSITIONS AND USE THEREOF

(75) Inventors: Gaylen M. Zentner, Salt Lake City, UT (US); Chung Shih, Sandy, UT (US); James C. McRea, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/692,655

(22) Filed: Mar. 28, 2007

Related U.S. Application Data
(63) Continuation of application No. 11/681,654, filed on Mar. 2, 2007.
Application No. 11/681,654 is a continuation of application No. PCT/US06/23566, filed on Jun. 16, 2006.
(60) Provisional application No. 60/691,362, filed on Jun. 16, 2005.

(51) Int. Cl. A61K 31/517; C07D 239/72

(52) U.S. Cl. 514/258.1; 544/283

(57) ABSTRACT

The present invention relates to pharmaceutical compositions and dosage compositions of compounds, including injectable formulations for the parenteral delivery of such compounds into patients in need of such treatment. Also featured are methods of making and using the compositions, including methods for the treatment of neoplastic diseases.

### CROSS REFERENCE TO RELATED U.S. APPLICATION

This application is a Continuation of U.S. patent application Ser. No. 11/681,654 filed on Mar. 2, 2007; which claims benefit to International Application PCT/US06/23566 filed Jun. 16, 2006; which claims benefit of U.S. Provisional Application Ser. No. 60/691,362, filed on Jun. 16, 2005, which is incorporated herein by reference in its entirety.

### FIELD OF THE INVENTION

The invention relates to pharmaceutical compositions and methods of making and using such compositions.

### BACKGROUND OF THE INVENTION

Various methods are available for administering therapeutic compounds to a patient. Such methods include, for example, parenteral, oral, ocular, nasal, buccal, transdermal, rectal, topical, and transmucosal administration. Variations of these different types of administrations exist. For example, parenteral administration includes intravenous, subcutaneous, intraperitoneal, intramuscular, intrathecal, intramedullary and intratumoral injection or infusion.

A chosen mode of administration may take into account various factors such as the disease that is being treated and the nature of the therapeutic compound. For example, one consideration that may be evaluated in selecting a route of administration is the bioavailability of the therapeutic compound after administration of the compound to the patient. Several factors can affect the bioavailability of the therapeutic compound such as solubility, aqueous solubility, stability, absorption, distribution, excretion/elimination, and metabolism of the compound.

Alternative formulations of compounds may also affect the relative bioavailability of the compound. For example, certain compounds are hydrophobic, thus exhibiting low aqueous solubility that is often accompanied by low bioavailability. Different techniques have been developed to increase bioavailability of compounds, such as solubilizing hydrophobic compounds in various vehicles. Accordingly, providing adequate bioavailability of therapeutic compounds through the use of appropriate formulations and routes of administration is desirable.

### BRIEF SUMMARY OF THE INVENTION

The present invention relates to pharmaceutical compositions comprising compounds having Formula I: 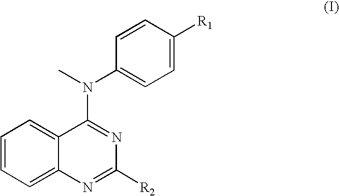
or a salt thereof wherein:

R₁ is chosen from OCH₃, OCHF₂, and OCH₂CH₃; and

R₂ is chosen from CH₃, Cl, CH₂F, and SCH₃;

and one or more liquid diluents. The present invention also relates to dosage compositions where the pharmaceutical composition has a compound to liquid diluent ratio sufficient to form a parenterally administrable dosage composition. For example, the dosage composition may be either the direct pharmaceutical composition or the pharmaceutical composition that has been further diluted with one or more liquid diluents. Such pharmaceutical compositions and dosage compositions may also contain one or more viscosity reducing agents, and/or excipients.

Compounds of Formula I are effective in inducing apoptosis in tumor cells and in treating cancer in animals, and are potentially effective in treating cancer or other hyperproliferative cellular disorders in mammals, particularly humans. When formulated as a dosage composition of the present invention and administered to a mammal, the compounds are sufficiently bioavailable to exert the desired pharmacological and clinical effect. Thus, the pharmaceutical compositions and dosage compositions allow for use of the compounds to treat diseases and disorders, such as neoplastic diseases, cancers, and diseases and disorders associated with the hyperproliferation of cells in mammals, particularly humans.

In one embodiment, one or more of the liquid diluents is aqueous. For example, an aqueous liquid diluent may be water, pharmaceutically acceptable aqueous solutions, aqueous saline solutions, Ringer's solutions, lactated Ringer's solutions, bicarbonate solutions, or aqueous dextrose solutions, or combinations thereof. The compositions of the invention may have concentrations of a compound of Formula I or a salt thereof in aqueous liquid diluents from about 0.01 μg/ml to about 150 mg/ml. Such compositions may also contain one or more excipients such as the antioxidant BHT (butylated hydroxytoluene).

In another embodiment, a liquid diluent is non-aqueous and comprises one or more surfactants, e.g., non-ionic surfactants. In general, the weight to weight ratio (w/w) between a compound of Formula I or a salt thereof and the non-ionic surfactant(s) may be from about 1:10,000 to about 1:1 (i.e. from about one gram of compound in 10,000 grams of surfactant to about one gram of compound in 1 gram of surfactant). For example, useful non-ionic surfactants can include a polyethoxylated castor oil, a polysorbate, a sorbitan ester, a polyoxyethylene fatty acid ester, a polyoxyethylene fatty acid ether, a polyoxyethylene alkyl ether, and an ethoxylated fatty acid. In specific embodiments, a polyethoxylated castor oil surfactant such as polyoxyl 35 castor oil is used in the non-aqueous liquid diluent, wherein the weight to weight ratio between a compound of Formula I or a salt thereof and polyoxyl 35 castor oil may be from about 1:500 to about 1:5. Optionally, the non-aqueous liquid diluent comprising one or more non-ionic surfactants also contains one or more viscosity reducing agents. For example, such viscosity reducing agents may be chosen from the pharmaceutically acceptable C₁₋₅ alkanols, benzyl alcohol, and low molecular weight aliphatic mono carboxylic acids. In a specific embodiment, the viscosity reducing agent is ethanol. In another specific embodiment, the ratio of non-ionic surfactant to viscosity reducing agent is from about 20:1 to about 1:10 (w/w).

In other embodiments, a liquid diluent may be a combination of aqueous diluents and non-aqueous diluents. For example, a non-aqueous liquid diluent comprising one or more non-ionic surfactants may further include an aqueous diluent, such as water, pharmaceutically acceptable aqueous solutions, aqueous saline solutions, Ringer's solutions, lactated Ringer's solutions, bicarbonate solutions, aqueous dextrose solutions, or combinations thereof. In a specific embodiment, the volume to volume ratio (v/v) of non-ionic surfactant to aqueous diluent may be from about 100:1 to about 1:20,000.

The invention also includes methods and kits to prepare and use the pharmaceutical compositions and/or dosage compositions. Kits of the invention may provide the compositions of the present invention in a variety of forms. For example, kits can provide at least one compound of Formula I or a salt thereof and at least one liquid diluent in a single container or in separate containers. Kits of the invention can also provide at least one compound of Formula I or a salt thereof and at least one liquid diluent in the same compartment or separate compartments of a single container. Kits may also include syringes, hypodermic needles, infusion sets and tubing apparatus, clamps, swabs, and other necessary or convenience peripherals to the parenteral administration process.

The invention includes various methods of preparing and using pharmaceutical compositions and/or dosage compositions. For example, the invention provides a method of preparing a pharmaceutical composition comprising at least partially dissolving at least one compound of Formula I in at least one pharmaceutically acceptable liquid diluent, such as at least one non-ionic surfactant (optionally combined with at least one viscosity reducing agent), and optionally adding at least one pharmaceutically acceptable aqueous diluent and/or at least one excipient, such as the antioxidant BHT.

The invention also includes various methods of administering the dosage compositions of the invention to a subject in need of treatment. A variety of diseases and disorders may be treated with pharmaceutical compositions of the invention, including cancer, neoplastic diseases and diseases and disorders associated with the hyperproliferation of cells in humans and animals. In one embodiment, pharmaceutical compositions of the invention can be administered by parenteral administration, such as intravenous, intraperitoneal, and intrathecal means. In another embodiment, pharmaceutical compositions of the invention can be administered directly to a desired location, such as by direct contact or direct injection into a tissue or tumor.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention, and the manner in which the same are accomplished, will be apparent from the following detailed description, and from the claims.

### DETAILED DESCRIPTION OF THE INVENTION

In accordance with the present invention, pharmaceutical compositions comprising compounds having Formula I: 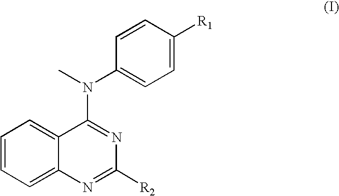
or a salt thereof wherein:

R₁ is chosen from OCH₃, OCHF₂, and OCH₂CH₃; and

R₂ is chosen from CH₃, Cl, CH₂F, and SCH₃;

and one or more liquid diluents. The present invention also relates to dosage compositions where the pharmaceutical composition has a compound to liquid diluent ratio sufficient to form a parenterally administrable composition.

Examples of such compounds include compounds and salts thereof chosen from: (2-Chloro-quinazolin-4-yl)-(4-methoxy-phenyl)-methyl-amine; (2-Fluoromethyl-quinazolin-4-yl)-(4-methoxy-phenyl)-methyl-amine; (4-Methoxy-phenyl)-methyl-(2-methyl-quinazolin-4-yl)-amine; (4-Difluoromethoxy-phenyl)-(2-methyl-quinazolin-4-yl)-methyl-amine; (2-Chloro-quinazolin-4-yl)-(4-ethoxy-phenyl)-methylamine; (4-Ethoxy-phenyl)-(2-methyl-quinazolin-4-yl)-methyl-amine; and (2-Methylthio-quinazolin-4-yl)-(4-methoxy-phenyl)-methyl-amine; which may be prepared according to methods disclosed in PCT Pub. No. WO2005003100, the relevant portions of which are incorporated herein by reference.

Particular compounds of the present invention have been discovered to exhibit low bioavailability when administered orally. Accordingly, in one aspect of the invention, compositions are provided that allow for therapeutic administration of compounds of the invention.

The amount of compound per unit volume of pharmaceutical composition and/or dosage composition may vary. For example, the amount of compound in the composition may be at least about 0.01 μg/ml or at least about 1 mg/ml. In another example, the amount of compound in the composition is between about 1 mg/ml and about 150 mg/ml. In other specific examples, the amount of compound in the pharmaceutical composition may be between about 1 mg/ml and about 50 mg/ml, or between about 5 mg/ml and about 15 mg/ml.

In one embodiment, a composition is provided wherein one or more liquid diluents are aqueous and comprise an aqueous diluent. Pharmaceutically acceptable aqueous diluents include solutions commonly used to prepare substances for parenteral administration, such as intravenous administration. Exemplary aqueous diluents include water, pharmaceutically acceptable aqueous solutions, saline solutions, aqueous dextrose solutions, such as dextrose 5% in water (D5W), Ringer's solutions, lactated Ringer's solutions, bicarbonate solutions, or combinations thereof.

Representative amounts of compound to aqueous diluents, namely (mg compound)/(ml aqueous diluent), include from about 0.01 μg/ml to about 150 mg/ml, from about 1 μg/ml to about 50 mg/ml, and from about 1 mg/ml to about 10 mg/ml.

In other embodiments, a composition is provided wherein one or more liquid diluents comprise one or more non-ionic surfactants. As used herein, the term “surfactant” refers to an agent that can solubilize compounds of the invention, and maintain solubilization once diluted into aqueous solutions. Exemplary surfactants are capable of completely solubilizing, or at least partially solubilizing the compounds of the invention and may form micelles or other self-associated structures when introduced into an aqueous environment.

Common examples of surfactants include potassium laurate, sodium alkylsulfates such as sodium dodecyl sulfate, hexadecyl sulphonic acid, and sodium dioctylsulphosuccinate, hexadecyl(cetyl)trimethylammonium bromide, dodecylpyridinium chloride, dodecylamine hydrochloride, N-dodecyl-N,N-dimethyl betaine, bile acids and salts, acacia, tragacanth, Igepal (polyoxyethylated nonylphenols), sorbitan esters (Spans), polysorbates (Tweens), Triton-X analogs (polyoxyethylated t-octylphenols), Brij analogs selected from the group consisting of polyoxyethylene lauryl ethers, polyoxyethylene cetyl ethers, polyoxyethylene stearyl ethers, and polyoxyethylene oleyl ethers, Myrj analogs (polyoxyethylene stearates), pluronics and tetronics selected from the group consisting of poloxamer and poloxamine type polyoxyethylene-polyoxypropylene derivatives, surface active drug agents such as phenothiazines and tricyclic antidepressants, and compounds and agents disclosed in Surfactants Systems, Their Chemistry, Pharmacy and Biology, by D. Attwood and A. T. Florence, (Chapman and Hall Pub. Co., 1983).

Although surfactants are amphipathic and can be anionic, cationic, or non-ionic, in exemplary embodiments of the present invention the surfactants are non-ionic. Pharmaceutically acceptable non-ionic surfactants typically include esters and ethers of polyoxyalkylene glycols, esters and ethers of polyhydric alcohols, or esters and ethers of phenols. Poloxamers and poloxamines are also examples of non-ionic surfactants. Specific examples of non-ionic surfactants include, but are not limited to, polyoxyethylene castor oil derivatives.

In certain embodiments, the one or more non-ionionic surfactants are chosen from: a polyethoxylated castor oil, a polysorbate, a sorbitan ester, a polyoxyethylene alkyl ether, a polyoxyethylene fatty acid ether, and an ethoxylated fatty acid. In specific embodiments, the non-ionic surfactant is a polyethoxylated castor oil, such as polyoxyl 35 castor oil. For example, the polyethoxylated castor oil may be CREMOPHOR® EL or CREMOPHOR® ELP (polyoxyl 35 castor oil; BASF, Ludwigshafen, Germany). CREMOPHOR® EL and CREMOPHOR® ELP are also known as macrogolglycerol ricinoleate or macrogolglyceroli ricinoleas. In other embodiments the non-ionic surfactant is an ethoxylated fatty acid, such as SOLUTOL® HS 15 (macrogol 15 hydroxystearate; BASF, Ludwigshafen, Germany). SOLUTOL® HS 15 is also known as ethoxylated 12-hydroxystearic acid or 12-hydroxystearic acid-polyethylene glycol copolymer.

Other exemplary non-ionic surfactants include polyoxyethylene 5 castor oil, polyethylene 9 castor oil, polyethylene 15 castor oil, d-alpha-tocopheryl polyethylene glycol succinate (TPGS), or monoglycerides, such as Myverol, glycerylmonooleate, monoolein, or aliphatic alcohol based nonionic surfactants, such as oleth-3, oleth-5, polyoxyl 10 oleyl ether, oleth-20, steareth-2, stearteth-10, steareth-20, ceteareth-20, polyoxyl 20 cetostearyl ether, PPG-5 ceteth-20, and PEG-6 capryl/capric triglyceride, Pluronic® copolymer non-ionic surfactants, such as Pluronic® L10, L31, L35, L42, L43, L44, L62, L61, L63, L72, L81, L101, L121, and L122, sorbitan fatty acid esters, such as Tween 20, Tween 40, Tween 60, Tween 65, Tween 80, Tween 81, and Tween 85, or, finally, ethoxylated glycerides, such as PEG 20 almond glycerides, PEG-60 almond glycerides, PEG-20 corn glycerides, and PEG-60 corn glycerides.

Surfactants are frequently characterized by a physical property known as the critical micelle concentration, or CMC. Values of CMCs are generally expressed in percent, representing the percentage concentration of an amphipathic molecule (i.e., surfactant) in an aqueous solution where micelles first form.

For example, in certain embodiments of the invention, non-ionic surfactants may be employed with CMC ranges of about 0.001% to about 0.5%, about 0.01% to about 0.10%, about 0.01% to about 0.05%, about 0.01% to about 0.04%, or about 0.01% to about 0.03%. Non-ionic surfactants with CMC numbers in this range have been found to provide a proper combination of physical characteristics, both in terms of solubilizing compounds of the invention, and in forming and maintaining micelles when the pharmaceutical composition is diluted into a larger volume of pharmaceutically acceptable aqueous diluent.

Surfactants may also be categorized and characterized by their hydrophilicity-lipophilicity balance number, or “HLB number.” The HLB system is a semi-empirical method used to predict what type of surfactant properties a particular molecular structure will provide. The HLB number of different surfactants can be used as a guide in the selection of a surfactant suitable for solubilizing a particular compound. Furthermore, HLB numbers are algebraically additive. Thus, by combining a surfactant with a low HLB number with a surfactant with a high HLB number, mixtures of surfactant can be prepared that exhibit HLB numbers intermediate between the two HLB numbers of the starting surfactants. The concept of HLB numbers is detailed in Remington's Pharmaceutical Sciences, 21^{st} Ed., Lippincott Williams & Wilkins (2006) pages 331-334.

For example, in the compositions of the invention, the HLB number of one or more surfactant(s) combined may be from between 10 and 14.

Examples of non-ionic surfactants that can be used in preparing the compositions of the instant invention particularly include the polyethoxylated castor oils. The term “ethoxylated castor oil,” as used above and herein, refers to castor oil that is modified with at least one oxygen-containing moiety. In particular the term refers to castor oil comprising at least one ethoxyl moiety. Furthermore, as used herein, the term polyoxyl 35 castor oil, which is also known as PEG-35 castor oil, macrogoglycerol ricinoleate and macrogolglyceroli ricinoleas, and alternatively as CAS Registry No. 61791-12-6, is a non-ionic surfactant, solubilizer and emulsifying agent used in the aqueous formulation of hydrophobic substances. Polyoxyl 35 castor oil is prepared by reacting castor oil with ethylene oxide in a molar ratio of 1:35. CREMOPHOR® EL and CREMOPHOR® ELP (BASF, Ludwigshafen, Germany) are polyoxyl 35 castor oil which have HLB numbers between 12 and 14, and critical micelle concentrations (CMC) of approximately 0.009% to 0.02%. CREMOPHOR® EL and CREMOPHOR® ELP have a density at 25° C. of 1.05-1.06 g/ml, and a viscosity of 600-800 mPa·s (See Product Literature on CREMOPHOR® EL and CREMOPHOR® ELP from BASF, the manufacturer).

Beneficially, surfactants used in embodiments of the invention may allow the compounds to be solubilized in, and delivered by way of, pharmaceutically acceptable aqueous diluents through self-association of the surfactant molecules, often in the form of micelles. Such final compositions with, for example, micelles in an aqueous diluent may be delivered by parenteral routes, especially via intravenous injection and infusion.

The amount of surfactant per unit volume of composition may vary. For example, a surfactant may makeup about 10 wt % to about 99.9 wt % of the pharmaceutical composition with the remainder made up of excipients, drug, stabilizing agents and the like. Exemplary ratios (weight/volume, i.e., weight of compound/volume of pharmaceutical composition) between compounds and the pharmaceutical composition may be from about 0.01 μg/ml or at least about 1 mg/ml. In another example, the amount of compound in the composition is between about 1 mg/ml and about 150 mg/ml. In other specific examples, the amount of compound in the pharmaceutical composition may be between about 1 mg/ml and about 50 mg/ml, or between about 5 mg/ml and about 15 mg/ml. In a specific embodiment, the non-ionic surfactant is polyoxyl 35 castor oil wherein the weight to weight ratio between compound of Formula I or a salt thereof, and the surfactant, is from about 1 mg/gram and about 50 mg/gram.

A composition of the present invention having one or more non-aqueous liquid diluents comprising one or more surfactants optionally may also include one or more viscosity reducing agents. For example, a liquid diluent comprising one ore more non-ionic surfactants may also contain one or more viscosity reducing agents.

As used herein, the term “viscosity reducing agent” means a pharmaceutically acceptable compound that, when mixed with a surfactant reduces the viscosity of the surfactant or liquid diluent to such an extent that the resulting solution can be readily handled by syringes and/or can be readily sterile filtered. Advantageously, viscosity reducing agents of the instant invention reduce the viscosity of the surfactant having a compound of Formula I dissolved therein to the point where the resulting solution can be filtered through sterile filters common to the art of sterile filtration pharmaceutical manufacturing processes, and that are often described as filters bearing pores of, for example, approximately 0.22 micrometers (μm) at room temperature. Such viscosity reducing agents allow for the use of surfactants that, by themselves, are too viscous to be readily handled by syringes and/or sterile filtered, in the compositions of the instant invention.

As used herein, the term “syringability” means the ability of a solution to be handled conveniently and accurately by a syringe fitted with hypodermic needles (e.g., 16 gauge to 30 gauge) at room temperature. Furthermore, “syringable solutions” can be readily handled, and volumetrically measured by means of a graduated syringe or other graduated measuring device.

The term “filterability,” as used herein, means the ability of a solution to be passed through a filter medium, and in the instant situation, means the ability of a solution to be passed through a filter commonly used in sterile filtration pharmaceutical manufacturing processes (typically described as having, for example, approximately 0.22 micrometer (μm) pores), to allow for the sterilization of the solution by the process of filtration. In particular, sterile filtration of the compositions of the instant invention can be achieved by passing compositions through a “sterile filter” with a pore size of approximately 0.22 micrometer (μm), or less. For example, sterile filtration of compositions of the instant invention can be achieved by passing these solutions through a polyvinylidene fluoride (PVDF) membrane with a pore size of 0.22 micrometer (μm), such as that found in Durapore™ filters (Millipore, Billerica, Mass., USA). With some compositions of the instant invention, sterile filtration is facilitated when the viscosity of the composition is lowered by the addition of at least one viscosity reducing agent.

In certain embodiments of the instant invention, such as those embodiments where a non-ionic surfactant used in the composition is a polyethoxylated castor oil, a viscosity reducing agent may be included in the composition to allow for convenient handling of solubilized compounds. In particular embodiments, at least one viscosity reducing agent and at least one surfactant are combined with a therapeutic compound to make a pharmaceutical composition. The weight/weight ratio, or alternatively the volume/volume ratio of surfactant to viscosity reducing agent can be adjusted so as to prepare a mixture that is a liquid at room temperature. For example, a pharmaceutical composition may be of sufficiently low viscosity that it can be readily transferred and measured by syringe, and can be readily sterile filtered through filters bearing pores of approximately 0.22 micrometers (μm), or less.

Representative viscosity reducing agents include alcohols such as ethanol, isopropanol, benzyl alcohol, or n-propyl alcohol, glycerol formal, N-methylpyrrolidone (NMP), dimethylsulfoxide (DMSO), dimethylformamide (DMF), polyethylene glycol 200 (PEG-200), polyethylene glycol 300 (PEG-300), polyethylene glycol 400 (PEG-400), propylene glycol, and water.

In specific embodiments, viscosity reducing agents are chosen from the pharmaceutically acceptable C₁₋₅ alkanols, benzyl alcohol, and low molecular weight aliphatic mono carboxylic acids. In particular embodiments, a chosen viscosity reducing agent is ethanol. In another specific embodiment the viscosity reducing agent is ethanol. Water also may have viscosity reducing utility in some embodiments.

In particular embodiments, a viscosity reducing agent may range from about 5 wt % to about 90 wt % of the pharmaceutical composition. In certain embodiments, the viscosity reducing agent is about 20 wt % to about 60 wt % of the composition. In other embodiments a viscosity reducing agent accounts for up to about 50 wt % of the composition.

As is true of each of the other constituents of the compositions of the present invention, the precise amount of viscosity reducing agent included in the pharmaceutical composition of the present invention may be varied. For example, the amount of viscosity reducing agent may vary to achieve a sought benefit in the syringability or filterability of the pharmaceutical composition. Exemplary ranges for viscosities in a composition having one or more surfactants, compounds and viscosity reducing agents, can be about 0.005 poise (0.0005 Pa·sec) to about 15.0 poise (1.5 Pa·sec) at about room temperature.

A composition of the present invention may optionally include excipients. For example, a liquid diluent comprising one or more non-ionic surfactants may also contain one or more excipients.

Exemplary excipients include such agents as preservatives, antioxidants, pH adjusting agents, osmolarity adjusting agents, and stabilizers. Preservatives are generally viewed as agents that prevent or inhibit microbial growth in a composition. Representative preservatives include parabens (e.g. methyl, ethyl, propyl, and butyl paraben), ethanol, isopropanol, sodium benzoate, benzyl alcohol, chlorobutanol, phenol, potassium sorbate, thimerosal, and benzalkonium chloride.

Antioxidants generally serve to protect the components of the compositions from oxidative damage. Examples of antioxidants include ascorbic acid, sodium ascorbate, ascorbyl palmitate, BHA (butylated hydroxyanisole), BHT (butylated hydroxytoluene), vitamin E, vitamin E PEG 1000, and TPGS (tocopherol polyethylene glycol succinate).

Excipients also include pharmaceutically acceptable pH adjusting agents and/or osmolarity adjusting agents. Such agents are used to improve the characteristics of the pharmaceutical composition so that it can be used to prepare solutions and liquids that are suitable for parenteral administration, especially intravenous injection and infusion. Suitable pH adjusting agents include buffers (e.g., phosphate, acetate, carbonate, tromethamine, citrate, lactate), acidifying agents (e.g., hydrochloric acid, phosphoric acid, tartaric acid, acetic acid, citric acid), and alkalinizing agents (e.g., sodium or potassium hydroxide, monoethanolamine, diethanolamine, triethanolamine). Examples of suitable osmolarity adjusting agents include any pharmaceutically acceptable water soluble compound, either ionic or nonionic in nature, such as glucose, sucrose, fructose, sodium chloride, sodium lactate, sorbitol, mannitol, glycerin, polyethylene glycols 400 to 4000, acidifying agents, alkalinizing agents, and pharmaceutically acceptable buffer salts.

In specific embodiments, a liquid diluent comprises at least one non-ionic surfactant and at least one antioxidant, such as BHT (butylated hydroxytoluene). In certain embodiments, a liquid diluent comprises at least one non-ionic surfactant and at least one aqueous diluent, such as water, saline, and/or aqueous dextrose solution. For example, the ratio of non-ionic surfactant to aqueous diluent may be from about 100:1 to about 1:20,000 (v/v). In other examples, the ratio of the non-ionic surfactant to aqueous diluent may also be at least about 1:1 (v/v), or from about 1:2 to about 1:1000 (v/v).

The compositions of the invention may also be a combination of one or more surfactants, viscosity reducing agents, excipients, and aqueous diluents. In a specific embodiment, the invention provides a dosage composition suitable for parenteral administration to a mammal, comprising a therapeutically effective amount of at least one compound having Formula I or a salt thereof, in admixture with a liquid diluent comprising polyoxyl 35 castor oil, ethanol, and an aqueous diluent selected from water, saline, and aqueous dextrose solution, wherein the weight ratio between said compound and said polyoxyl 35 castor oil is from about 1:500 to about 1:5, the weight ratio between said polyoxyl 35 castor oil and said ethanol is from about 1:10 to about 20:1, and the v:v ratio between said polyoxyl 35 castor oil and said aqueous diluent is from about 1:50 to about 1:5,000.

The invention also includes methods and kits to prepare and use the pharmaceutical compositions and/or dosage compositions. Kits of the invention may provide the compositions of the present invention in a variety of manners. Kits can provide components of pharmaceutical compositions of the invention together in a single container or compartment or in separate containers or compartments. For example, kits can provide at least one compound of Formula I and at least one liquid diluent in a single container or in separate containers. Kits of the invention can also provide at least one compound of Formula I and at least one liquid diluent in the same compartment or separate compartments of a single container. For example, compounds of Formula I, aqueous diluents, and optionally one or more viscosity reducing agents, surfactants, and/or excipients may be provided separately in a kit or various combinations may be provided together. Containers may be configured in such a manner that liquids can be conveniently introduced into or removed by way of syringes or other devices that move liquids or provide pathways for the movement of liquids. For example, at least a portion of a container may be made of a material that can be punctured by a syringe needle. Optionally, kits may include instructions for preparation and or use of pharmaceutical compositions and/or dosage compositions.

In a specific example, a kit provides a vial or bottle containing a pharmaceutical composition of the invention. Alternatively, components of a pharmaceutical composition of the invention may be provided in a kit. For example, a kit may provide a vial or bottle containing at least one compound of Formula I and another vial or bottle containing at least one liquid diluent. In specific embodiments, a liquid diluent comprises an aqueous diluent, such as water, saline, or aqueous dextrose solution. In other embodiments a liquid diluent comprises a non-ionic surfactant, such as a polyethoxylated castor oil, a polysorbate, a sorbitan ester, a polyoxyethylene fatty acid ester, a polyoxyethylene fatty acid ether, a polyoxyethylene alkyl ether, or an ethoxylated fatty acid. A liquid diluent may optionally include a non-ionic surfactant. For example, a liquid diluent comprising a non-ionic surfactant may include a viscosity reducing agent, such as the pharmaceutically acceptable C₁₋₅ alkanols, benzyl alcohol, and low molecular weight aliphatic mono carboxylic acids.

A kit may also provide components of a pharmaceutical composition of the invention in other manners. For example, a kit may provide at least one compound of Formula I in admixture with a viscosity reducing agent, and also provide a surfactant and/or an aqueous diluent. Alternatively, kits may provide separately a compound of Formula I, a non-ionic surfactant, a viscosity reducing agent, and/or an aqueous diluent. Kits may also include excipients. For example, an excipient may be provided separately from other components of the pharmaceutical composition, as part of a liquid diluent, or in admixture with a compound of Formula I.

In some embodiments, a kit may also optionally provide pre-treatment medicinal agents. Such pre-treatment medicinal agents may alleviate or reduce side-effects associated with administration of pharmaceutical and/or dosage compositions. For example, such pre-treatment medicinal agents may be antihistamines, anti-inflammatory steroids such as glucocorticoids, and/or combinations thereof.

Instructions may be provided describing how to appropriately combine the compound of Formula I and other components of the kit. Kits may additionally contain items such as filtration devices, sterile filtration devices and intravenous injection bags (i.v. bags). Examples of suitable i.v. bags include polyolefin-lined i.v. bags such as PAB® bags manufactured by B. Braun Medical, Inc., of Bethlehem, Pa., U.S.A.

The invention includes various methods of preparing and using pharmaceutical compositions. For example, the invention provides a method of preparing pharmaceutical compositions comprising at least partially dissolving at least one compound of Formula I in one ore more liquid diluents. In a specific method, pharmaceutical compositions are prepared by forming an emulsion or a miceller solution with one or more liquid diluents and at least one compound of Formula I. In another method, pharmaceutical compositions are prepared by simply dissolving at least one compound of Formula I in one ore more liquid diluents. In yet another method, pharmaceutical compositions are prepared by forming a suspension of at least one compound of Formula I in one or more liquid diluents.

A pharmaceutical composition may be volumetrically measured and transferred by syringe, which may optionally include a sterilization step, such as by sterile filtration techniques or other sterilization techniques such as heat exposure, e-beam irradiation, or gamma ray irradiation. For example, the pharmaceutical composition may be filter sterilized by passing the composition through a suitable sterile filtration device with a filtration pore size of approximately 0.22 μm, or less. In certain embodiments, the pharmaceutical composition is passed through a sterile filtration device as it is being delivered into a pharmaceutically acceptable aqueous diluent to prepare a dosage composition. The reagents can be provided in one or more containers suitable for administration by intravenous injection. For example, a composition of at least one compound of Formula I and at least one non-ionic surfactant can be introduced directly into a suitable aqueous diluent contained in an i.v. bag, whereupon the two liquids are combined to form an injectable dosage composition.

In specific embodiments, a liquid diluent comprises an aqueous diluent, such as water, saline, or aqueous dextrose solution. In other embodiments a liquid diluent comprises a non-ionic surfactant, such as a polyethoxylated castor oil, a polysorbate, a sorbitan ester, a polyoxyethylene fatty acid ester, a polyoxyethylene fatty acid ether, a polyoxyethylene alkyl ether, or an ethoxylated fatty acid. A liquid diluent may optionally include a non-ionic surfactant. For example, a liquid diluent comprising a non-ionic surfactant may include a viscosity reducing agent, such as the pharmaceutically acceptable C₁₋₅ alkanols, benzyl alcohol, and low molecular weight aliphatic mono carboxylic acids.

For example, in one embodiment a method for preparing pharmaceutical compositions comprises: (a) combining a quantity of at least one compound of Formula I in a volume of at least one non-ionic surfactant and, optionally, at least one pharmaceutically acceptable viscosity reducing agent; (b) combining a measured volume of the liquid of (a) into a volume of at least one aqueous diluent. In some embodiments, at least one excipient, such as BHT, is also included in a pharmaceutical composition of the invention. Combined liquids and/or compounds can be mixed by means such as simple inversion and/or agitation. The methods of preparing compositions of the invention can be scaled to any volume desired.

The invention also includes various methods of administering the dosage compositions of the invention to a mammal, such as a human. A variety of diseases and disorders may be treated with dosage compositions of the invention, including neoplastic diseases, such as cancer. The dosage compositions can also be used in the treatment of other hyperproliferative diseases and disorders, including psoriasis, epidermal hyperproliferation, restenosis, vascular proliferative diseases such as diabetic retinopathy, and diabetic complications. In addition, the dosage compositions can be used as immunosuppressants and can treat disorders of the immune system, including autoimmune diseases.

In specific embodiments, the invention includes a method of treating neoplastic diseases, such as cancer, comprising administering to a subject in need of treatment a dosage composition comprising at least one compound of Formula I and a liquid diluent. In some embodiments, the administration of a dosage composition is preceded by the administration of one or more pre-treatment medicinal agents. Such pre-treatment medicinal agents may be administered by any effective dosage routes, such as parenteral or oral routes. Pre-treatment medicinal agents may alleviate or reduce side-effects associated with administration of pharmaceutical and/or dosage compositions. For example, such pre-treatment medicinal agents may be antihistamines, anti-inflammatory steroids such as glucocorticoids, and/or combinations thereof.

In specific embodiments, a liquid diluent comprises an aqueous diluent, such as water, saline, or aqueous dextrose solution. In other embodiments a liquid diluent comprises a non-ionic surfactant, such as a polyethoxylated castor oil, a polysorbate, a sorbitan ester, a polyoxyethylene fatty acid ester, a polyoxyethylene fatty acid ether, a polyoxyethylene alkyl ether, or an ethoxylated fatty acid. A liquid diluent may optionally include a non-ionic surfactant. For example, a liquid diluent comprising a non-ionic surfactant may include a viscosity reducing agent, such as C₁₋₅ alkanol, benzyl alcohol, and low molecular weight aliphatic mono carboxylic acids.

Various methods are available for administering therapeutic compounds to a patient. Such methods include, for example, parenteral, oral, ocular, nasal, buccal, transdermal, rectal, topical, and transmucosal administration. In certain embodiments, compositions of the invention can be administered by parenteral administration, such as intravenous, subcutaneous, intraperitoneal, intramuscular, intrathecal, intramedullary and intratumoral injection. Compositions of the invention can also be administered directly to a desired location, such as by direct contact or direct injection to a tissue.

The amount and dosage of compounds and compositions to be administered can be adjusted to achieve the desired therapeutic effect. For example, compounds in the dosage compositions of the invention can be effective at amounts of from about 0.05 mg to about 4000 mg per day, from about 0.1 mg to about 2000 mg per day, and from about 1 mg to about 100 mg per day. The composition dosages may be administered at one time either rapidly or over a predetermined period of time, or may be divided into a number of doses to be administered at predetermined intervals of time.

It should be understood that the dosage ranges set forth above are exemplary only and that the amount of compounds to be administered can vary with various factors such as the body weight or body surface area of the patient treated, the state of disease conditions, the activity of the compound, the stability of the compound in the patient's body, the route of administration, the ease of absorption, distribution, and excretion of the compound by the body, the age and sensitivity of the patient to be treated, and the like, as will be apparent to a skilled artisan.

The following examples will serve to illustrate various aspects and/or features of the invention and are not to be regarded as limitations of the scope of the invention.

### EXAMPLE 1

### Pharmaceutical Composition

A pharmaceutical composition is prepared by combining and mixing 100 grams of (4-Methoxy-phenyl)-methyl-(2-methyl-quinazolin-4-yl)-amine hydrochloride and 1 gram of BHT and dissolving into 10 liters of D5W with the pH adjusted to pH=5 with hydrochloric acid. This solution is sterile filtered using a 0.2 μm Teflon filter (PTFE).

### EXAMPLE 2

### Pharmaceutical Kit

A pharmaceutical kit is prepared that contains 100 mg of (4-Methoxy-phenyl)-methyl-(2-methyl-quinazolin-4-yl)-amine hydrochloride powder in a first glass vial, and a second vial containing 10 ml of sterile D5W with the pH adjusted to pH=5 with hydrochloric acid. A solution is prepared by transferring the pH=5 D5W with a syringe and 20 gauge hypodermic needle into the vial containing the drug powder. The drug is dissolved under mild agitation to form a solution.

### EXAMPLE 3

### Pharmaceutical Composition

A pharmaceutical composition was formed by dissolving 300.1 grams (4-Methoxy-phenyl)-methyl-(2-methyl-quinazolin-4-yl)-amine hydrochloride into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### EXAMPLE 4

### Pharmaceutical Composition

A pharmaceutical composition was formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### EXAMPLE 5

### Pharmaceutical Composition

A pharmaceutical composition is formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 11.652 kg viscosity reducing agent (ethanol 190 proof), and 2 kg WFI (water for injection). This solution is sterile filtered through a 0.2 μm Millipore Durapore filter (PVDF), and packaged into 10 ml sterile glass vials.

### EXAMPLE 6

### Method of Administration

About 0.01 ml to about 50 ml of the pharmaceutical composition of Example 5 is accurately measured and then added to an i.v. bag containing about 100 ml to about 1000 ml of sterile dextrose 5% in water (D5W). The amount of pharmaceutical composition and D5W used varies according to the desired therapeutic dose and size of the patient. The resulting mixture is then parenterally infused into the patient.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

1. A pharmaceutical composition, comprising:
a therapeutically effective amount of at least one compound having Formula I: 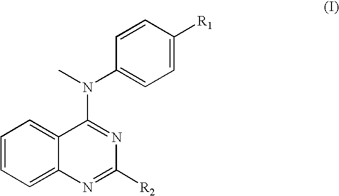
or a salt thereof wherein:
R₁ is chosen from OCH3, OCHF2, and OCH2CH3; and
R₂ is chosen from CH3, Cl, CH2F, and SCH3;
and one or more liquid diluents.

2. The pharmaceutical composition of claim 1, wherein one or more of the liquid diluents comprise one or more aqueous diluents.

3. The pharmaceutical composition of claim 2, wherein the at least one compound is in admixture with the one or more liquid diluents at amounts of from about 0.01 μg/ml to about 150 mg/ml.

4. The pharmaceutical composition of claim 2, wherein said aqueous diluents are chosen from: water, saline solutions, Ringer's solutions, lactated Ringer's solutions, bicarbonate solutions, and aqueous dextrose solutions.

5. The pharmaceutical composition of claim 1, wherein one or more of said liquid diluents comprise one or more non-ionic surfactants.

6. The pharmaceutical composition of claim 5, wherein the weight to weight ratio between said at least one compound and said one or more liquid diluents is from about 1:10,000 to about 1:1.

7. The pharmaceutical composition of claim 5, wherein said one or more non-ionic surfactants are chosen from:
a polyethoxylated castor oil,
a polysorbate,
a sorbitan ester,
a polyoxyethylene fatty acid ester,
a polyoxyethylene alkyl ether,
a polyoxyethylene fatty acid ether, and
an ethoxylated fatty acid.

8. The pharmaceutical composition of claim 5, wherein one or more of said non-ionic surfactants is a polyethoxylated castor oil.

9. The pharmaceutical composition of claim 8, wherein the polyethoxylated castor oil is polyoxyl 35 castor oil.

10. The pharmaceutical composition of claim 9, wherein the weight to weight ratio between said at least one compound and said polyoxyl 35 castor oil is from about 1:500 to about 1:5.

11. The pharmaceutical composition of claim 5, wherein said one or more liquid diluents further comprise at least one viscosity reducing agent.

12. The pharmaceutical composition of claim 11, wherein the at least one viscosity reducing agent is chosen from C₁₋₅ alkanols, benzyl alcohol, and low molecular weight aliphatic mono carboxylic acids.

13. The pharmaceutical composition of claim 11, wherein at least one of the viscosity reducing agents is ethanol.

14. The pharmaceutical composition of claim 13, wherein the ratio of the at least one non-ionic surfactant to the at least one viscosity reducing agent is about 20:1 to about 1:10 (w/w).

15. The pharmaceutical composition of claim 5, further comprising one or more excipients.

16. The pharmaceutical composition of claim 15, wherein the one or more excipients are chosen from preservatives, antioxidants, pH adjusting agents, osmolarity adjusting agents, and stabilizers.

17. The pharmaceutical composition of claim 16, wherein at least one of the excipients is an antioxidant.

18. The pharmaceutical composition of claim 5 further comprising at least one aqueous diluent.

19. The pharmaceutical composition of claim 18, wherein the volume to volume ratio of the one or more non-ionic surfactants to the at least one aqueous diluent is 100:1 to 1:20,000.

20. The pharmaceutical composition of claim 19, wherein said ratio is from about 1:50 to about 1:5,000.

21. The pharmaceutical composition of claim 18, wherein the at least one aqueous diluent is chosen from water, saline solutions, Ringer's solutions, lactated Ringer's solutions, bicarbonate solutions, and aqueous dextrose solutions.

22. The pharmaceutical composition of claim 21, further comprising at least one viscosity reducing agent.

23. The pharmaceutical composition of claim 22, wherein the at least one viscosity reducing agent is chosen from C₁₋₅ alkanols, benzyl alcohol, and low molecular weight aliphatic mono carboxylic acids.

24. The pharmaceutical composition of claim 23, wherein the at least one viscosity reducing agent is ethanol.

25. The pharmaceutical composition of claim 23, further comprising one or more excipients.

26. The pharmaceutical composition of claim 25, wherein the one or more excipients are chosen from preservatives, antioxidants, pH adjusting agents, osmolarity adjusting agents, and stabilizers.

27. The pharmaceutical composition of claim 26, wherein one or more of the excipients is an antioxidant.

28. The pharmaceutical composition according to claim 27, wherein the compound of Formula I is chosen from:
(2-Chloro-quinazolin-4-yl)-(4-methoxy-phenyl)-methyl-amine;
(2-Fluoromethyl-quinazolin-4-yl)-(4-methoxy-phenyl)-methyl-amine;
(4-Methoxy-phenyl)-methyl-(2-methyl-quinazolin-4-yl)-amine;
(4-Difluoromethoxy-phenyl)-(2-methyl-quinazolin-4-yl)-methyl-amine;
(2-Chloro-quinazolin-4-yl)-(4-ethoxy-phenyl)-methylamine;
(4-Ethoxy-phenyl)-(2-methyl-quinazolin-4-yl)-methyl-amine;
(2-Methylthio-quinazolin-4-yl)-(4-methoxy-phenyl)-methyl-amine; and
pharmaceutically acceptable salts thereof.

29. A kit comprising the pharmaceutical composition of claim 1.

30. The kit of claim 29 wherein components of the pharmaceutical composition are provided together in a single container or compartment.

31. The kit of claim 29 wherein pre-treatment medicinal agents are optionally included.

32. The kit of claim 31 wherein the pre-treatment medicinal agents are chosen from antihistamines, anti-inflammatory steroids, and/or combinations thereof.

33. (canceled)

34. A method of treating diseases and disorders chosen from neoplastic diseases, cancers, disorders of the immune system, and diseases and disorders associated with the hyperproliferation of cells in mammals, comprising administering to a mammal in need of such treatment a pharmaceutical composition according to claim 1.

35. A dosage composition comprising a pharmaceutical composition according to claim 1 diluted in one or more liquid diluents.

36. A dosage composition suitable for parenteral administration to a mammal, comprising a therapeutically effective amount of at least one compound having Formula I: 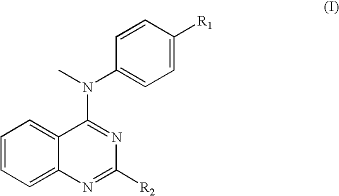
or a salt thereof wherein:
R₁ is chosen from OCH3, OCHF2, and OCH2CH3; and
R₂ is chosen from CH3, Cl, CH2F, and SCH3;
in admixture with a liquid diluent comprising
polyoxyl 35 castor oil,
ethanol,
an antioxidant, and
an aqueous diluent selected from water, saline solutions, Ringer's solutions, lactated Ringer's solutions, bicarbonate solutions, and aqueous dextrose solutions, wherein the weight to weight ratio between said compound and said polyoxyl 35 castor oil is from about 1:500 to about 1:5, the weight to weight ratio between said polyoxyl 35 castor oil and said ethanol is from about 1:10 to about 20:1, and the volume to volume ratio between said polyoxyl 35 castor oil and said aqueous diluent is from about 1:50 to about 1:5,000.

37. The dosage composition according to claim 36, wherein the compound of Formula I is chosen from:
(2-Chloro-quinazolin-4-yl)-(4-methoxy-phenyl)-methyl-amine;
(2-Fluoromethyl-quinazolin-4-yl)-(4-methoxy-phenyl)-methyl-amine;
(4-Methoxy-phenyl)-methyl-(2-methyl-quinazolin-4-yl)-amine;
(4-Difluoromethoxy-phenyl)-(2-methyl-quinazolin-4-yl)-methyl-amine;
(2-Chloro-quinazolin-4-yl)-(4-ethoxy-phenyl)-methylamine;
(4-Ethoxy-phenyl)-(2-methyl-quinazolin-4-yl)-methyl-amine;
(2-Methylthio-quinazolin-4-yl)-(4-methoxy-phenyl)-methyl-amine; and
pharmaceutically acceptable salts thereof.

38. A method of preparing the dosage composition of claim 35 comprising the steps of:
a. at least partially dissolving at least one compound of Formula I in one or more liquid diluents to form a pharmaceutical composition; and
b. diluting said pharmaceutical composition in one or more liquid diluents.

39. (canceled)

40. A method of treating diseases and disorders chosen from neoplastic diseases, cancers, disorders of the immune system, and diseases and disorders associated with the hyperproliferation of cells in mammals, comprising administering to a mammal in need of such treatment a pharmaceutical composition according to claim 1.

41. The use of claim 40, wherein the dosage composition is administered parenterally to a mammal in need thereof.

42. A kit comprising the dosage composition of claim 35.

43. The kit of claim 42 wherein components of the dosage composition are provided together in a single container or compartment.

44. The kit of claim 42 wherein pre-treatment medicinal agents are optionally included.

45. The kit of claim 44 wherein the pre-treatment medicinal agents are chosen from antihistamines, anti-inflammatory steroids, and/or combinations thereof.
